(19) United States
(12) Patent Application Publication
(10) Pub. No.: US 2013/0072532 A1
(43) Pub. Date: Mar. 21, 2013

(54) TOPICAL TRANSDERMAL DEXMEDETOMIDINE COMPOSITIONS AND METHODS OF USE THEREOF

(71) Applicants: Geraldine A. Henwood, Malvern, PA (US); Randall J. Mack, Paoli, PA (US); Christopher T. Sharr, Malvern, PA (US); John J. Koleng, JR., Austin, TX (US)

(72) Inventors: Geraldine A. Henwood, Malvern, PA (US); Randall J. Mack, Paoli, PA (US); Christopher T. Sharr, Malvern, PA (US); John J. Koleng, JR., Austin, TX (US)

(73) Assignee: Recro Pharma, Inc., Malvern, PA (US)

(21) Appl. No.: 13/520,959

(22) PCT Filed: Jan. 7, 2011
(86) PCT No.: PCT/US2011/020462

§ 371 Date: Dec. 3, 2012

Related U.S. Application Data
(60) Provisional application No. 61/293,440, filed on Jan. 8, 2010.

(51) Int. Cl. A61K 31/4174; A61P 29/00

(52) U.S. Cl. 514/396

(57) ABSTRACT

Analgesic topical formulations of dexmedetomidine and methods of use thereof in the treatment and management of pain and other conditions.

### CROSS-REFERENCE TO RELATED APPLICATIONS

This application claims the priority of U.S. Provisional Patent Application No. 61/293,440, filed on Jan. 8, 2010, which is incorporated herein by reference in its entirety.

### BACKGROUND

Dexmedetomidine, 5-[(1S)-1-(2,3-dimethylphenyl)ethyl]-1H-imidazole, is a non-narcotic α₂-adrenoceptor agonist with sedative and analgesic properties.

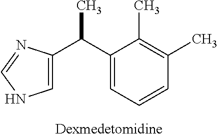

Currently, dexmedetomidine is only commercially available as an injectable formulation indicated for sedation, and it must be administered intravenously by a heath care professional. Although dexmedetomidine has analgesic properties, a formulation useful as an analgesic, however, is not commercially available. Moreover, for a variety of reasons the commercially available injectable formulation is not suitable for use as an analgesic that can be self-administered. A continuing and unmet need exists for dexmedetomidine-based analgesic medicines that, for example, may be self-administered to produce analgesia (or otherwise treat or prevent pain) without sedation.

### SUMMARY

This application describes analgesic, transdermal formulations of dexmedetomidine, pharmaceutically acceptable salts thereof, and derivatives thereof, as well as methods of use thereof.

Provided herein are new analgesic, transdermal formulations of dexmedetomidine and/or a pharmaceutically acceptable salt thereof, and/or a derivative thereof, and methods of use thereof in the treatment or prevention of pain. Such pharmaceutical compositions include dexmedetomidine or a pharmaceutically acceptable salt or derivative (e.g., pro-drug) thereof, such as in an amount sufficient to produce analgesia (e.g., treat or prevent pain) and a pharmaceutically acceptable transdermal delivery vehicle, as well as optional additional ingredients, such as, but not limited to, viscosity adjusters, pH adjusters, preservatives, excipients, emulsifiers, buffers, colorants, and the like. The dexmedetomidine or its pharmaceutically acceptable salt or derivative (e.g., pro-drug) thereof, in the pharmaceutically acceptable transdermal vehicle may be packaged into a suitable container or device.

In an exemplary embodiment, a method for administering dexmedetomidine or a pharmaceutically acceptable salt or derivative thereof to a mammal includes applying a predetermined quantity of a transdermal composition to the skin of the mammal, the composition comprising dexmedetomidine or a pharmaceutically acceptable salt or derivative thereof in a pharmaceutically acceptable transdermal delivery vehicle in an effective amount to provide transdermal absorption of a pharmaceutically effective amount of the dexmedetomidine through the skin of the mammal into the systemic circulatory system of the mammal. Upon transdermal absorption, the dexmedetomidine produces analgesia in the mammal, and without sedation when dosed appropriately.

Additional features may be understood by reference to the following detailed description and examples.

### BRIEF DESCRIPTION OF THE DRAWINGS

FIG. 1 graphically illustrates observed versus predicted plasma concentration-time curves at steady-state after once daily doses in humans using exemplary formulations hereof.

FIG. 2 graphically illustrates observed versus predicted steady-state plasma concentration-time curves after twice-daily dosing in humans using exemplary formulations hereof.

FIG. 3 graphically illustrates predicted results of multiple day dosing humans once daily using an exemplary formulation hereof.

FIG. 4 graphically illustrates predicted results of multiple day dosing humans twice-daily using an exemplary formulation hereof.

FIG. 5 graphically illustrates dose results using Formula 1-85 in Dogs.

FIG. 6 graphically illustrates dose results using Formula 1-53 in Dogs.

FIG. 7 graphically illustrates dose results using Formula 1-83 in Dogs.

### DETAILED DESCRIPTION

Provided herein are new analgesic, transdermal formulations of dexmedetomidine or pharmaceutically acceptable salts or derivatives thereof and methods of use thereof in the treatment and management of pain.

Dexmedetomidine is a specific α2-adrenergic receptor agonist that causes sedation, anesthesia, and analgesia in mammals. In humans, dexmedetomidine is commercially available for sedation of initially intubated and mechanically ventilated patients during treatment in an intensive care setting, as well as sedation of non-intubated patients prior to or during surgical and other procedures. See, e.g., U.S. Pat. Nos. 6,716,867 and 6,313,311.

The pharmacokinetics of dexmedetomidine in humans have been studied after intravenous (i.v.), intramuscular (i.m.), and transdermal administration. The mean elimination half-life is 1.5 to 3 h after i.v. and i.m. dosing, respectively, and 5.6 h after transdermal administration. After i.m. and transdermal administration, the time to maximum concentration in blood is 1.6-1.7 h and 6 h, respectively, and the absolute bioavailability has been estimated to be 73% and 88%, respectively. See, e.g., “Pharmacodynamics and pharmacokinetics of intramuscular dexmedetomidine,” Scheinin et al., Clin. Pharmacol. Ther. 52, 53-46 (1992); “The pharmacokinetics and hemodynamic effects of intravenous and intramuscular dexmedetomidine hydrochloride in adult human volunteers,” Dyck et al., Anesthesiology 78, 813-20 (1993); and “Pharmacokinetics and pharmacodynamics of transdermal dexmedetomidine,” Kivistö et al., Eur. J. Clin. Pharmacol. 46, 345-49 (1994).

Dexmedetomidine is also known to be absorbed from other tissues, such as the oral cavity. After buccal administration in which human subjects held a solution of dexmedetomidine in the mouth without swallowing, the mean buccal bioavailability has been measured at 81.8%, with a maximum concentration at approximately 1.5 h and an apparent elimination half-life of 1.9 h. See, e.g., “Bioavailability of dexmedetomidine after extravascular doses in healthy subjects,” Anttila et al., Br. J. Clin. Pharmacol. 56, 691-93 (2003).

According to the present invention, dexmedetomidine may be administered to an animal or human subject for the purpose of ameliorating, managing, curing, preventing, or otherwise treating pain. In an exemplary embodiment, a method for administering dexmedetomidine or a pharmaceutically acceptable salt or derivative thereof to a mammal includes applying to the skin of a mammal a predetermined dosage of a transdermal composition comprising dexmedetomidine or a pharmaceutically acceptable salt or derivative thereof in a pharmaceutically acceptable transdermal vehicle in an effective amount to provide absorption of a pharmaceutically effective amount of the dexmedetomidine through the skin of the mammal into the systemic circulatory system of the mammal. Upon transdermal absorption, the dexmedetomidine produces analgesia in the mammal.

For example, many patients with cancer and other ailments continue to experience moderate to severe pain despite chronic analgesic therapy, and this can occur as intermittent breakthrough pain often due to increases in a patient's activity level. Attempts to counteract this type of pain by increasing the dose of long-acting formulations of analgesics often produce slow onset of analgesia and unwanted side-effects of sedation, constipation or nausea and vomiting, especially with opioid analgesics. However, the analgesic, transdermal formulations of dexmedetomidine described herein provide potent non-narcotic analgesic effects, and preferably sustained, prolonged, controlled analgesic effects, and combinations thereof to ameliorate, manage, cure, prevent, or otherwise treat pain. Treatment may include the use of the transdermal dexmedetomidine formulations alone, or in combination with other dexmedetomidine formulations (e.g., sublingual, transmucosal, injectable, and/or other analgesic compositions (e.g., NSAIDS, narcotics, and the like).

The dexmedetomidine products described herein are transdermal pharmaceutical formulations for the treatment of pain. As used herein, the term “pharmaceutically acceptable” includes those compounds, materials, compositions, dosage forms, packages, and methods of use thereof that are within the scope of sound medical judgment and suitable for use in contact with the tissues of human beings and animals without excessive toxicity, irritation, allergic response, or other problem or complication, while being commensurate with a reasonable benefit/risk ratio and eliciting a desired pharmacological response.

Dexmedetomidine contains a basic nitrogen atom capable of forming pharmaceutically acceptable salts with pharmaceutically acceptable acids. The term “pharmaceutically acceptable salts” in this respect refers to the relatively non-toxic, inorganic, and organic acid addition salts of dexmedetomidine. These salts may be prepared in situ during final isolation and purification of dexmedetomidine or by separately reacting purified dexmedetomidine, in its free base form with a suitable organic or inorganic acid, and thereafter isolating the salt thus formed. Furthermore, the salt may be formed during a manufacturing process to produce the transdermal formulation. Representative pharmaceutically acceptable salts include the hydrohalide (including hydrobromide and hydrochloride), sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, 2-hydroxyethylsulfonate, and laurylsulphonate salts, and the like. See, e.g., “Pharmaceutical Salts,” Berge et al., J. Pharm. Sci. 66, 1-19 (1977). Dexmedetomidine hydrochloride is an example of a pharmaceutically acceptable salt. Use of dexmedetomidine hydrochloride may be preferable to the use of dexmedetomidine per se in the transdermal formulations described herein because, in some cases, the hydrochloride salt has greater water solubility and stability against oxidation by ambient oxygen.

Dexmedetomidine derivatives may include covalent modifications that create a pro-drug. Upon administration, the pro-drug derivative undergoes chemical modification by the mammal that yields dexmedetomidine. Pro-drugs may be used to favorably alter the biodistribution or the pharmacokinetics of dexmedetomidine or to produce other desirable characteristics. For example, a reactive nitrogen of dexmedetomidine may be derivatized with a functional group that is cleaved, enzymatically or non-enzymatically, reductively, oxidatively, or hydrolytically, to reveal the active pharmaceutical ingredient. Uses of certain types of pro-drugs are known (see, e.g., R. B. Silverman, 1992, “The Organic Chemistry of Drug Design and Drug Action,” Academic Press, Chp. 8). For example, pro-drugs may be prepared in situ during the final isolation and purification of the compounds, or by separately reacting the purified compound in its free base form with a suitable derivatizing agent.

The dexmedetomidine transdermal compositions include one or more pharmaceutically acceptable carriers such as gels, creams, or liquids by 30% to about 99.995% by weight. These carriers may be solvents, cosolvents, or non-solvents for dexmedetomidine or its pharmaceutically acceptable salts or derivatives thereof. Suitable materials are liquids or flowable compositions at room temperature and remain in that state at room temperature, preferably at both ambient pressure as well as under elevated pressure. Useful liquids and flowable compositions are not particularly restricted, provided they do not interfere with the desirable medical use of the compositions, and they carry a therapeutically useful amount of dexmedetomidine or a pharmaceutically acceptable salt or derivative thereof (e.g., dexmedetomidine hydrochloride). Examples of pharmaceutically acceptable liquids include water, ethanol, dimethylsulfoxide, propylene glycol, polyethylene glycol, propylene carbonate, pharmaceutically acceptable oils (e.g., soybean, sunflower, peanut, etc.) and the like. Examples of flowable compositions include creams, gels, suspensions, lotions, serums, ointments, and the like. The pharmaceutically acceptable liquid or flowable composition is selected either to dissolve the active pharmaceutical ingredient, to produce a stable, homogenous suspension of it, or to form any combination of a suspension, solution, mixtures or other pharmaceutically compatible compositions.

In addition to the foregoing constituent ingredients, transdermal formulations of dexmedetomidine may include one or more excipients other than the pharmacologically active drug, which are included in the manufacturing process or are contained in a finished pharmaceutical product dosage form. Examples of excipients include viscosity modulating materials (e.g. polymers, sugars, sugar alcohols, gums, clays, silicas, and the like (e.g., polyvinylpyrrolidone (PVP)) (from about 0.01% to about 65% by weight). Other examples of excipients include preservatives (e.g., ethanol, benzyl alcohol, propylparaben and methylparaben) (from about 0.001% to about 20% by weight). Still other examples of excipients include buffers and pH-adjusting agent (e.g., sodium hydroxide, citrate, and citric acid) (from about 0.01% to about 5% by weight). Coloring agents (from about 0.001% to about 5% by weight), fragrances (from about 0.001% to about 1% by weight), chelating agents (e.g., EDTA) (from about 0.001% to about 1% by weight), UV absorbers (from about 0.001% to about 10% by weight), among others, are additional examples of suitable excipients. Suitable transdermal formulations are further detailed in the Examples further set forth herein.

Additionally, methods of making and using such formulations will be apparent to one skilled in the art pursuant to the teachings herein. For example, transdermal dexmedetomidine formulations may be made by mixing appropriate quantities of the ingredients described herein in accordance with standard recognized good manufacturing practices. Such excipients may be included in the formulation to improve patient or subject acceptance, to improve bioavailability, to increase shelf-life, to reduce manufacturing and packaging costs, to comply with requirements of governmental regulatory agencies, and for other purposes. The relative amounts of each ingredient should not interfere with the desirable pharmacological and pharmacokinetic properties of the resulting formulation.

The analgesic, transdermal formulations of dexmedetomidine described herein are intended for administration directly to the skin and/or the mucosa (e.g., the oral, nasal, rectal, or genital mucosa) in a mammal. Drug delivery occurs substantially via the transdermal route—as opposed to swallowing followed by gastrointestinal absorption. The term “transdermal’ refers to delivery across or through a skin membrane. The term “transmucosal” refers to delivery across or through a mucosal membrane. In particular, “oral transmucosal” delivery of a drug includes delivery across any tissue of the mouth, pharynx, larynx, trachea, or upper gastrointestinal tract, particularly the sublingual, buccal, gingival and palatal mucosal tissues.

Transdermal delivery allows a drug substance access to the systemic blood circulation, thereby providing for direct systemic administration independent of gastrointestinal influences and avoiding undesirable first-pass hepatic metabolism. As compared to other routes of administration, transdermal absorption of dexmedetomidine in the present formulations may have a significantly more controllable and prolonged duration with desirable bioavailability, even over multiple days of dosing. Additionally, because first-pass metabolism is bypassed, the total amount of active pharmaceutical ingredient in the formulation may be reduced, thereby reducing the likelihood of deleterious side effects (e.g., hypotension, sedation) and providing a cost benefit to the manufacturer.

The transdermal formulation may be administered to mammals, including humans, as well as human companion animals (e.g., cats, dogs), agricultural livestock, and other animals in need thereof. One will appreciate that administering conventional dosage forms such as tablets, capsules, syrups, etc. or injectable analgesic formulations to non-human animals is often problematic, and the transdermal formulations described herein are especially useful in the treatment of such animals. The use of transdermal delivery has the same advantages over other delivery methods in young children and the elderly.

“Analgesia” is the alleviation or elimination of the sensation of pain. As used herein, “pain” encompasses a wide range of clinical manifestations, and it has a broad meaning. Pain perception is highly subjective, and different people experience pain in different ways and with greatly different intensities. The International Association for the Study of Pain defines pain as “an unpleasant sensory and emotional experience associated with actual or potential tissue damage, or described in terms of such damage.” More simply stated, pain includes any sensory experience that causes suffering and is associated with an unpleasant awareness of one's own body. Non-limiting types and causes of pain include neuralgia, myalgia, hyperalgesia, hyperpathia, neuritis, and neuropathy. Pain is often a symptom of an underlying physiological abnormality, such as cancer or arthritis. Some types of pain have no clearly identified causes, such as migraine headache pain. Pain may also be caused by physical trauma, such as burns or surgery. Viral infections, such as Herpes zoster (chicken pox and shingles), can also cause pain. Withdrawal from chemical dependence on alcohol or drugs of abuse is also often associated with pain symptoms. Accordingly, “pain” is understood herein to have a very broad meaning and its claimed uses should not be construed as being limited to any particular malady or condition.

“Sedation” as used herein means depressed consciousness in which a patient or subject retains the ability to independently and continuously maintain an open airway and a regular breathing pattern, and to respond appropriately and rationally to physical stimulation and verbal commands. As used herein “without sedation” means that the Patient experiences a level of sedation not greater than Level 3 on the Ramsay Sedation Scale, in other words, the Patient is either at Level: Level 1=anxious, agitated, or restless; Level 2=cooperative, oriented, and tranquil; or Level 3=sedated but responds to commands. “Significant sedation” as used herein means that the patient or subject experiences sedation of Level 4 or greater on the Ramsay Sedation Scale, wherein Level 4=asleep; brisk response to light glabellar tap or loud auditory stimulus; Level 5=asleep; sluggish response to light glabellar tap or loud auditory stimulus; Level 6=asleep; no response to painful stimulus. “Significant sedation” as used herein is also consistent with a patient's self-evaluation on the Stanford Sleepiness Scale, with Subject patients rating their degree of sedation as greater than or equal to Level 3, wherein: Level 1=Feeling active, vital, alert, or wide awake; Level 2=Functioning at high levels, but not at peak; able to concentrate; Level 3=Awake, but relaxed; responsive but not fully alert; Level 4=Somewhat foggy, let down; Level 5=Foggy; losing interest in remaining awake; slowed down; Level 6=Sleepy, woozy, fighting sleep; prefer to lie down; or Level 7 No longer fighting sleep, sleep onset soon; having dream-like thoughts.

The formulations of dexmedetomidine described herein may be co-administered with other pain-treatment medicines, including NSAIDS such as aspirin, ibuprofen, naproxen, celecoxib, acetaminophen, and other cyclooxygenase inhibitors; opioids such as codeine, oxycodone, morphine, methadone, and fentanyl; anticonvulsants and antiarrhythmics such as phenytoin and carbamazepine; and antidepressants such as amitriptyline, imipramine, and venlafaxine. Such co-administration may be contemporaneous, wherein dexmedetomidine and another pain-treatment medicine are both administered at the same time. Alternatively, because of the rapid-acting nature of the transdermal compositions described herein, a patient may be administered a shorter or longer acting pain medicine on a regular schedule, with transdermal dexmedetomidine being administered as needed throughout the day or from time to time as required. In some cases, the dosage of the alternative pain-treatment medicine may be reduced because of a beneficial synergistic effect produced by dexmedetomidine, which supplements the primary pharmacological therapy. In particular, dexmedetomidine may significantly potentiate the effectiveness of opioids, permitting a reduction in required opioid dosage while maintaining equivalent therapeutic usefulness.

Analgesic, transdermal formulations of dexmedetomidine, or a pharmaceutically acceptable salt or derivative thereof are preferably provided in metered dosages so that a predetermined amount of the active pharmaceutical ingredient is properly administered to the subject in a pharmaceutically effective amount. For example, the transdermal formulation may be provided in individual dose packaging or in multiple packaged units, for instance, as a bulk container containing multiple doses in a system comprising a sealed container fitted with a metering pump. Typically, a human patient is treated by topical self-administration of one or more actuations from the pump. Each actuation of the pump provides a metered dose. An advantage of delivery is the ability to titrate patients by single doses as required through single, discrete applications. This advantage is typically absent from other forms of drug delivery (e.g., patches, lozenges, tablets, and suppositories) in which a one-size-fits-all dosage is administered in a standard regimen. Additional advantages of topical transdermal formulations include their ease of use, especially when self-administered absent an attending health care professional.

Pump action dispensers are characterized in requiring the application of external pressure for actuation, for example, external manual, mechanical or electrically initiated pressure. This is in contrast to pressurized systems, e.g., propellant-driven aerosol or compressed gas sprays, where actuation is typically achieved by controlled release of pressure e.g., by controlled opening of a valve. In certain embodiments, pump formulations are preferred as the use of a pump spray with the formulations herein allows for the administration of a known quantity and a controllable size of formulation. In other embodiments, pressurized systems containing a reservoir of pressurized propellant gas (e.g., carbon dioxide, nitrogen, chlorofluorocarbons, hydrofluoroalkanes, etc.) may produce suitable dispensed amounts. In certain preferred embodiments, the viscosity of the delivered formulations further provides for an increase in surface area by being spread evenly across the skin or mucosa as opposed to being rubbed on the skin or mucosa in a thicker layer (e.g., a gel versus a cream). The density (e.g., cream versus foam versus gel) may contribute to a desired pattern and thickness of the composition on the skin or mucosa.

The spray pump device may be premetered or, alternatively, the device may be device-metered. Premetered devices preferably contain previously measured doses or a dose fraction in some type of units (e.g., single unit dose amount of solution, single or multiple blisters or other cavities) that may be included in the device during manufacture or by the patient before use. Typical device-metered units have a reservoir containing formulation sufficient for multiple doses that are delivered as metered sprays by the device itself when activated by the patient. The device may be metered both in the amount of drug substance delivered (i.e., the dosage per actuation), as well as the length of time between each dosage. Limiting the time between each dosage can prevent over-use by limiting how often a dosage can be delivered to the patient.

Manufacturing considerations include the reproducibility of the formulation and any dispensing mechanism or transdermal delivery vehicle. Maintaining the reproducibility of these parameters through the expiration dating period and ensuring the functionality of the device (e.g., spray mechanism, electronic features, sensors, and the like) through its lifetime under patient-use conditions is important as any alteration in these parameters could lead to variability in dosing and absorption, which could lead to potential side effects and decreased therapeutic usefulness.

The administered dose of the formulation by any device may be dependent on the design, reproducibility, and performance characteristics of the container closure system. A suitable device that provides the desired distribution is an important factor for the correct performance of the dexmedetomidine product. Actuation parameters (e.g., force, speed, hold and return times) should also be considered with respect to the device. Moreover, the device should be compatible with formulation components. Furthermore, the device should be designed to prevent partial metering, as well as over metering, of the dexmedetomidine formulation, including the dexmedetomidine, pharmaceutically acceptable salt thereof, or derivative thereof, when used according to patient instructions for use.

A typical formulation device includes a base unit, a discharge actuator, an orifice for the formulation to be released from the device, and a reservoir. Preferably the reservoir is filled with the drug substance and other excipients (e.g., liquid vehicle, excipients, etc. as discussed elsewhere herein) prior to dispensing to the patient, e.g., at the manufacturing site. The reservoir preferably defines a measured amount of dexmedetomidine, pharmaceutically acceptable salt thereof, or derivative thereof to be discharged upon activation. The reservoir body may be any acceptable material, for example, formed simply by a section of a cylindrical hollow of a plastic, steel, such as stainless steel, transparent material, or the like so that its production is very simple. An actuator, which is movable relative to the orifice for activating discharge, may be provided on or with the device. In the course of the actuating movement, the reservoir opens, e.g., by puncturing, to administer a single dosage through an orifice. During a part of the actuating travel following the starting position, an elevated pressure is built up. In a subsequent portion of the actuating movement continuing in the same direction, the medium may be relieved of the pressure at one of the sides and communicated to an orifice. In such a manner, the medium is pushed from the reservoir and through the orifice by the action of pressure.

Typically, as any sprayable transdermal formulation leaves the orifice, the droplets follow a trajectory which is influenced by the orifice shape, as well as by pressure asserted. In some embodiments, the droplet size, spray geometry, and the spray pattern are dependent on the design of the pump and/or the properties of the formulation. In certain embodiments, the orientation of the actuator, pump design, and the properties of the formulation will influence the spray symmetry and the shape. The spray pattern may also be optimized to disperse the droplets over a wider pathway thereby increasing the surface area on the skin through which the compound can be absorbed. Any such spray device may further be designed to facilitate ease of patient use and placement of the administered spray to specific regions of the skin.

### EXAMPLES

The following transdermal formulations of dexmedetomidine were prepared as examples. Several of the exemplary formulations were tested in mammals, as described herein.

### Example 1a

### Placebo Transdermal Gel Formulation (Carrier)

Exemplary ingredients and methods to prepare 50 g of Methyl Salicylate/Menthol Hydroalcoholic Clear Gel.

TABLE A
50 g of Methyl Salicylate/Menthol Hydroalcoholic
Clear Gel (RECRO1-45)

| | | | | | |
|---|---|---|---|---|---|
| Phase & | | | | | |
| Ingredient | | | g/ | g/ | |
| No. | Material | % w/w | 150 g | 250 g | Lot |

Phase A

| | | | | |
|---|---|---|---|---|
| 1 | Methyl Salicylate | 15 | 22.50 | XU0224 |
| 2 | Menthol | 7 | 10.50 | |
| 3 | Carbopol 981A | 0.3 | 0.45 | EC884CD234 |
| 4 | Alcohol 200 | 15 | 22.50 | XX3061 |
| | Proof | | | |
| 5 | Propylene Glycol | 20 | 30.00 | XJ0809 |
| | Sub-total | | 85.95 | |

Phase B

| | | | | | |
|---|---|---|---|---|---|
| 6 | Klucel HF | 0.6 | 0.90 | 1.50 | 89442 |
| 7 | Alcohol 200 | 20 | 30.00 | 50.00 | XX3061 |
| | Proof (same | | | | |
| | questions) | | | | |
| 8 | Purified Water | 21.5 | 32.25 | 53.75 | |
| | Sub-total | | 63.15 | | |

Phase C

| | | | | |
|---|---|---|---|---|
| 9 | Trolamine | Qs to | 3.598 g | XT0007 |
| | | pH 8 | | |
| | Total | 99.4 | | |
| | Purified Water | qs to | | |
| | | 100% | | |

TABLE B
Method for 50 g of Methyl Salicylate/Menthol
Hydroalcoholic Clear Gel (RECRO1-45)

| Steps | Description |
|---|---|
| A | In a first suitable container, dissolve ingredient No. 2 (menthol) |
| | in ingredient No. 1 (methyl salicylate) in a suitable container. |
| B | Add ingredient No. 3 (Carbopol) to the result of step A |
| C | Add ingredient No. 4 (alcohol) and ingredient No. 5 to the result |
| | of Step B, and mix and disperse to form PART A. |
| D | To form PART B, in a suitable second container, disperse |
| | Ingredient No. 6 (Klucel HF) in Ingredient No. 7 (alcohol) and mix |
| | well, such as by using a homogenizer. |
| E | Add Ingredient No. 8 (water) to the result of Step D and |
| | homogenize to obtain a clear, viscous solution referred to as PART |
| | B. |
| F | Add PART A to PART B and mix well Note: prepare PART B in |
| | excess and pour off the required quantity into PART A. |
| G | Slowly add Ingredient No. 9 (trolamine) to the result of Step F |
| | until pH of the final composition is about 8. |

In this example, the inventor(s) noted that: 1) pH=0.592 g, and a white mass precipitated on water addition; and 2) addition of water to rehydrate or reconstituted gel results in precipitate as well. The precipitate effect is believed to result from methyl salicilate being insoluble as more water is added. Alternatively or additionally, the precipitation may be at least in part pH dependent.

### Example 1b

### Transdermal Dexmedetomidine Gel Formulation

TABLE C
50 g of Gel Formulation at [0.5 mg/g Dex
HCl] Concentration (RECRO1-53)

| Ingredient | | |
|---|---|---|
| No. | Material | g/50 g |
| 1 | Methyl Salicylate/Menthol Hydroalcoholic | 49.975 g |
| | Clear Gel of Example 1 (Lot Recro 1-45) | |
| 2 | Dexmedetomidine HCl | 0.025 g |

TABLE D
Methods for 50 g of Gel Formulation at [0.5
mg/g Dex HCl] Concentration (RECRO1-53)

| Steps | Description |
|---|---|
| A | Dispense ingredient No. 1 into 100 ml glass beaker |
| B | Add ingredient No. 2 |
| C | Mix thoroughly |
| D | Package the formulation from Step C into suitable |
| | containers, such as glass scintillated vials |
| E | Seal package from step D and label as appropriate. |

In this exemplary method, several observations were made at Step B (upon introduction of Ingredient No. 2 DEXMEDETOMIDINE HCl: 1) the gel formulation became clouded; and 2) the resulting gel of Step B had a decreased viscosity as compared to the gel of Step A. To address observation 1 concerning cloudiness, trolamine was added and was effective. In this example, trolamine was added at the rate of 0.463 g trolamine (identified as lot XT0007) to 32.532 g of the gel resulting from Step C.

### Examples 2, 3 & 4

### Dex HCl Topical Cream Formulations

The base placebo cream formulations were used to make active dexmedetomidine HCl transdermal formulations as described further herein.

### Example 2a

### Placebo O/W Cream Base Formulation (Carrier)

TABLE D2
Oil and Cream Base Formulation (RECRO 1-88)

| | | | |
|---|---|---|---|
| No. | Material Description | % (w/w) | g/175 g |

Water Phase

| | | | |
|---|---|---|---|
| 1 | Dexmedetomidine HCl | 0.00 | |
| 2 | Imidurea | 0.50 | |
| 3 | Dibasic Sodium Phosphate Heptahydrate | 0.50 | |
| 4 | Polysorbate 80 | 1.00 | |
| 5 | Purified Water | 48.00 | |
| 6 | Propylene Glycol | 20.00 | |
| | 70/30 Water/Oil Phase Ratio | | 122.50 |

Oil Phase

| | | | |
|---|---|---|---|
| 7 | ST-Cyclomethicone 5 | 12.00 | 21.00 |
| 8 | Light Mineral Oil | 12.00 | 21.00 |
| 9 | Stearyl Alcohol | 2.00 | 3.50 |
| 10 | Cetyl Alcohol | 2.00 | 3.50 |
| 11 | Span 80 | 2.00 | 3.50 |
| | Total | 100.00 | 52.50 |

TABLE D3
Methods for Oil and Cream Base Formulation (RECRO 1-88)

| | |
|---|---|
| Steps | Description |

Water Phase

| | |
|---|---|
| A | Mix the required quantity of 2, 3, 4, and 67.2 g of Purified Water. |
| B | Adjust the pH of A to 9.0 (+/−0.2) with 2 N HCl or 2 N NaOH. |
| | Quantity added: 0.66 g |
| C | Add any remaining purified water as 16.8 g - Quantity of 2 N |
| | HCl or 2 N NaOH. |
| D | Mix in required quantity of 6. Heat with stirring to 50-60° C. |

Oil Phase

| | |
|---|---|
| E | Mix the required quantity of 7, 8, 9, 10, and 11 using heat. |
| F | Heat the mixture to approximately 50-60° C. to match the |
| | aqueous phase. |

Combination

| | |
|---|---|
| G | Prepare and hold each phase individually. |
| H | Pour oil phase into water phase with continuous mixing. |

### Example 2b

### Transdermal Dexmedetomidine Cream Formulation

TABLE E
Dex HCl Topical Cream 0.5 mg/g (RECRO2-1)

| Ingredient | | | | |
|---|---|---|---|---|
| No. | Material | % | g/50 g | Lot # |
| 1 | Placebo O/W Cream | 99.95 | 49.975 g | RECRO1-88 |
| | Base Formulation of | | | |
| | Example 2a | | | |
| | (RECRO1-88) | | | |
| 2 | Dexmedetomidine | 0.05 | 0.025 g | 1224654 |
| | HCl | | | |
| | Total | 100.000 | | |

TABLE F
Methods for Dex HCl Topical Cream 0.5 mg/g (RECRO2-1)

| Steps | Description |
|---|---|
| A | Dispense ingredient No. 1 into 100 ml glass beaker |
| B | Add ingredient No. 2 |
| C | Mix thoroughly |
| D | Package the formulation from Step C into suitable |
| | containers, such as 20 ml glass scintillated vials |
| E | Seal package from step D and label as appropriate. |

### Example 3a

### Placebo O/W Cream Base Formulation (Carrier)

TABLE F1
Oil and Water Cream Base (RECRO 1-98)

| | | | | |
|---|---|---|---|---|
| No. | Material Description | % (w/w) | g/125 g | Lot No. |

Water Phase

| | | | | |
|---|---|---|---|---|
| 1 | Dexmedetomidine HCl | 0.00 | 0.00 | |
| 2 | Imidurea | 0.50 | 0.63 | YA0770 |
| 3 | Dibasic Sodium Phosphate | 0.50 | 0.63 | E49160 |
| | Heptahydrate | | | |
| 4 | Polysorbate 80 | 1.50 | 1.88 | XV0879 |
| 5 | Purified Water | 47.50 | 59.38 | N/A |
| 6 | Transcutol P | 15.00 | 18.75 | 450739011 |
| 7 | Propylene Glycol | 5.00 | 6.25 | XV0820 |
| | 70/30 Water/Oil Phase Ratio | | 87.50 | |

Oil Phase

| | | | | |
|---|---|---|---|---|
| 8 | Light Mineral Oil | 14.50 | 18.13 | XB0680 |
| 9 | ST-Cyclomethicone | 10.00 | 12.50 | 0005665349 |
| 10 | Stearyl Alcohol | 2.00 | 2.50 | XU0571 |
| 11 | Cetyl Alcohol | 2.00 | 2.50 | YQ3022 |
| 12 | Span 80 | 1.50 | 1.88 | YM0729 |
| | Total | 100.00 | 37.50 | |

TABLE F2
Methods to Make Oil and Water Cream Base (RECRO 1-98)
Preparation
Water Phase

| A | Mix the required quantity of 2, 3, 5, | Initial pH | Final pH |
|---|---|---|---|
| | and 53.4 g of Purified Water | 7.99 | 9.02 |

| | |
|---|---|
| B | Adjust the pH of (A) to 9.0 (+/−0.2) with 2 N HCl or 2 N |
| | NaOH. Quantity added: 0.490 g |
| C | Add any remaining purified water as 5.98 g - Quantity of 2 N |
| | HCl or 2 N NaOH. |
| D | Mix in required quantity of 6 and 7. Heat with stirring to 50-60° C. |

Oil Phase

| | |
|---|---|
| E | Mix the required quantity of 8, 9, 10, 11, and 12 using heat. |
| F | Heat the mixture to approximately 50-60° C. to match the |
| | aqueous phase. |

Combination

| | |
|---|---|
| G | Prepare and hold each phase individually. |
| H | Pour oil phase into water phase with continuous mixing. |

Water Phase = 51° C. at time of mixing
Oil Phase = 56° C. at time of mixing
Cools to a white cream

### Example 3b

### Transdermal Dexmedetomidine Cream Formulation

TABLE G
Ingredients for Dex HCl Topical Cream 0.5 mg/g (RECRO2-2)

| Ingredient | | | | |
|---|---|---|---|---|
| No. | Material | % | g/50 g | Lot # |
| 1 | Placebo O/W Cream | 99.95 | 49.975 g | RECRO1-98 |
| | Base Formulation of | | | |
| | Example 3a | | | |
| | (RECRO1-98) | | | |
| 2 | Dexmedetomidine | 0.05 | 0.025 g | 1224654 |
| | HCl | | | |
| | Total | 100.000 | | |

TABLE H
Methods for Dex HCl Topical Cream 0.5 mg/g (RECRO2-2)

| Steps | Description |
|---|---|
| A | Dispense ingredient No. 1 into 100 ml glass beaker |
| B | Add ingredient No. 2 |
| C | Mix thoroughly |
| D | Package the formulation from Step C into suitable |
| | containers, such as 20 ml glass scintillated vials |
| E | Seal package from step D and label as appropriate. |

### Example 4a

### Placebo O/W Cream Base Formulation (Carrier)

TABLE H1
Oil and Water Cream Base (RECRO 1-100)

| | | | | |
|---|---|---|---|---|
| No. | Material Description | % (w/w) | g/175 g | Lot No. |

Water Phase

| | | | | |
|---|---|---|---|---|
| 1 | Dexmedetomidine HCl | 0.00 | 0.00 | |
| 2 | Imidurea | 0.40 | 0.70 | YA0770 |
| 3 | Dibasic Sodium Phosphate | 0.40 | 0.70 | E49160 |
| | Heptahydrate | | | |
| 4 | Polysorbate 80 | 1.00 | 1.75 | XV0879 |
| 5 | Purified Water | 48.20 | 84.35 | N/A |
| 6 | Propylene Glycol | 10.00 | 17.50 | XV0820 |
| | 60/40 Water/Oil Phase Ratio | | 105.00 | |

Oil Phase

| | | | | |
|---|---|---|---|---|
| 7 | ST-Cyclomethicone | 10.00 | 17.50 | 0005665349 |
| 8 | Isopropyl Myristate | 8.00 | 14.00 | XT0205 |
| 9 | Light Mineral Oil | 15.00 | 26.25 | XB0680 |
| 10 | Stearyl Alcohol | 2.50 | 4.38 | XU0571 |
| 11 | Cetyl Alcohol | 2.50 | 4.38 | YQ3022 |
| 12 | Span 80 | 2.00 | 3.50 | YM0729 |
| | Total | 100.00 | 52.50 | |

TABLE H2
Methods to Make Oil and Water Cream Base (RECRO 1-100)
Preparation
Water Phase

| | | | |
|---|---|---|---|
| A | Mix the required quantity of 2, 3, 5, | Initial pH | Final pH |
| | and 75.9 g (90% of total) of Purified | 7.95 | 9.02 |
| | Water | | |

| | |
|---|---|
| B | Adjust the pH of (A) to 9.0 (+/−0.2) with 2 N HCl or 2 N |
| | NaOH. Quantity added: 0.572 g |
| C | Add any remaining purified water as 8.45 g - Quantity of 2 N |
| | HCl or 2 N NaOH. |
| D | Mix in required quantity of 6. Heat with stirring to 50-60° C. |

Oil Phase

| | |
|---|---|
| E | Mix the required quantity of 7, 8, 9, 10, and 11 using heat. |
| F | Heat the mixture to approximately 50-60° C. to match the |
| | aqueous phase. |

Combination

| | |
|---|---|
| G | Prepare and hold each phase individually. |
| H | Pour oil phase into water phase with continuous mixing. |

Water = 50° C. at time of mixing
Oil = 60° C. at time of mixing
Sets up to a smooth white cream

### Example 4b

### Transdermal Dexmedetomidine Cream Formulation

TABLE I
Dex HCl Topical Cream 0.5 mg/g (RECRO2-3)

| Ingredient | | | | |
|---|---|---|---|---|
| No. | Material | % | g/50 g | Lot # |
| 1 | Placebo O/W Cream | 99.95 | 49.975 g | RECRO1-100 |
| | Base Formulation of | | | |
| | Example 2a | | | |
| | (RECRO1-100) | | | |
| 2 | Dexamedetomidine | 0.05 | 0.025 g | 1224654 |
| | HCl | | | |
| | Total | 100.000 | 50.00 | |

TABLE J
Methods for Dex HCl Topical Cream 0.5 mg/g (RECRO2-3)

| Steps | Description |
|---|---|
| A | Dispense ingredient No. 1 into 100 ml glass beaker |
| B | Add ingredient No. 2 |
| C | Mix thoroughly |
| D | Package the formulation from Step C into suitable |
| | containers, such as 20 ml glass scintillated vials |
| E | Seal package from step D and label as appropriate. |

### Example 5a

### Placebo O/W Silicone Base Formulation (Carrier)

TABLE J1
Recro 1-86 - Oil and Water Silicone Emulsion (RECRO 1-85)
(O/W) Silicone Emulsion, pH 8.5 - 2

| | | | | |
|---|---|---|---|---|
| No. | Material | % (w/w) | g/100 g | Lot |

Phase A

| | | | | |
|---|---|---|---|---|
| 1 | Cyclomethicone 5-NF | 10.00 | 10.00 | 0005665349 |
| 2 | Light Mineral Oil | 10.00 | 10.00 | XB0680 |
| 3 | Dimethiconol Blend 20% | 5.00 | 5.00 | 0005483185 |
| 4 | Silky Wax 10 | 2.00 | 2.00 | 0005699369 |
| 5 | Emulsifier 10 | 2.00 | 2.00 | 0005605454 |

Phase B

| | | | | |
|---|---|---|---|---|
| 6 | Imidurea | 0.50 | 0.50 | YA0770 |
| 7 | Propylene Glycol | 10.00 | 10.00 | XJ0809 |
| 8 | 25 mM PBS, pH 8.5 | 60.50 | 60.50 | Recro 1-73 |
| | Total | 100.0 | | |

TABLE J2
Method to Make Recro 1-86 - Oil and
Water Silicone Emulsion (RECRO 1-85)

| Steps | Description |
|---|---|
| A | Combine 1, 2, 3, and 4 and heat to obtain a solution. |
| B | Add 5 to A. Continue to mix - Phase A. |
| C | Combine 6, 7, 8 - Phase B |
| D | Add Phase B to A with mixing. |

Smooth, white emulsion formed

### Example 5b

### Transdermal Dexmedetomidine Cream Formulation

TABLE J3
Dexmedetomidine HCl 0.5 mg/g Topical
Silicone Emulsion (RECRO 1-85)

| No. | Material | % (w/w) | g/50 g | Lot |
|---|---|---|---|---|
| 1 | Dexmedetomidine HCl | 0.05 | 0.025 | 1224654349 |
| 2 | Silicone Emulsion Base, | 99.95 | 49.975 | Recro 1-86 |
| | pH 8.5 | | | |
| | Total | 100.000 | 50.000 | |

TABLE J4
Method to Make Dexmedetomidine HCl 0.5 mg/g
Topical Silicone Emulsion (RECRO 1-85)

| Steps | Description |
|---|---|
| A | Dispense 2 into 100 mL glass beaker. |
| B | Dispense 1 and add to A. |
| C | Mix thoroughly. |
| D | Package into 20 mL glass scintillation vials. |
| E | Label |

Smooth, white emulsion formed

Several of the exemplary formulations herein were administered to canines to determine absorption and other performance characteristics as a topical transdermal delivery composition for dexmedetomidine. Of those compositions tested, the composition identified herein as Recro 1-85, produced desirable systemic levels of dexmedetomidine. Those formulations are suitable for use in, at least, treatment of canine pain and associated conditions and may also be suitable for use in other mammals, including humans. The foregoing examples are merely exemplary and are not intended as limiting the scope of the inventions herein. Several of the exemplary topical transdermal formulations were tested in animals and others developed and/or tested included alcoholic gels, oil, and water emulsion creams, among others. The relevant results of relevant animal testing are summarized herein.

### CLINICAL EXAMPLES

### Clinical Study Period 1 (REC-10-006)

Based upon the successful experiments involving transdermal formulations in animals, tests and trials in humans were developed and conducted for several formulations and methods of administration and treatment. The human formulations derived from the exemplary animal study formulations but were made in conformity with cGMP requirements and other regulatory requirements for human use. Several exemplary embodiments of the topical transdermal formulations and methods herein have been utilized in humans, in early stage clinical trials (aka Phase I studies). The exemplary formulations used in those clinical trials to date are as follows:

TABLE K
DEX-TD.02

| Item | | | | Target |
|---|---|---|---|---|
| Code | Material | Grade | Formula | Weight (g) |
| XX-2005 | Dexmedetomidine | n/a | 0.1000 | 0.50 |
| | Free Base | | | |
| XX-2006 | Dimethyl Suloxide | USP | 45.0000 | 225.00 |
| X5-1551 | Ethanol 200 Proof | USP | 20.0000 | 100.00 |
| X18 | Purified Water | USP | 18.9000 | 94.50 |
| X5-1019 | Propylene Glycol | USP | 10.0000 | 50.00 |
| XX-1070 | Sorbitol | NF | 5.0000 | 25.00 |
| X5-1088 | Hydroxypropyl | NF | 1.000 | 5.00 |
| | Cellulose | | | |
| | | Total | 100.000% | 500.00 g |

TABLE L
DEX-TD.03

| Item | | | | Target |
|---|---|---|---|---|
| Code | Material | Grade | Formula | Weight (g) |
| XX-2005 | Dexmedetomidine | n/a | 0.10 | 0.50 |
| | Free Base | | | |
| XX-2007 | Laurocapram (Azone, | n/a | 3.00 | 15.00 |
| | Water Soluble) | | | |
| X5-1551 | Ethanol 200 Proof | USP | 20.00 | 100.00 |
| X18 | Purified Water | USP | 20.90 | 104.50 |
| X5-1019 | Propylene Glycol | USP | 50.00 | 250.00 |
| XX-1070 | Sorbitol | NF | 5.00 | 25.00 |
| X5-1088 | Hydroxypropyl | NF | 1.00 | 5.00 |
| | Cellulose | | | |
| | | Total | 100.000% | 500.00 g |

Pain Management in Humans.

Since the year 2000, when the Joint Commission revised their standards for the assessment and management of pain, the treatment of pain has taken an increasingly significant position in medical care. Often referred to as the “fifth vital sign”, subjects must now be routinely evaluated for pain symptoms so that therapy may be appropriately adjusted. While this increased focus has brought more attention to the issue of patient comfort and quality of life, the available range of tools has remained largely the same. Current medications run the gamut in duration of activity, ranging from acute medications that provide relief for 1-2 hours, to alternative formulations which can provide as much as 72 hours of analgesia. At the same time, these dosage forms commonly rely on a similar set of active ingredients, which often work through the same opioid pathway to provide relief (morphine, oxycodone, fentanyl). The result, while there is generally not a ceiling on the effect provided by opiate medications, the use of high doses, and multiple opiate medications may lead to an increased occurrence of adverse events which may force a decision between symptoms and side effects.

Dexmedetomidine is a selective α2-aderenoceptor agonist, with selectivity approximately 10-fold that of clonidine. Dexmedetomidine has an extensive history of safe intravenous use in acute and surgical settings, utilizing its sedative properties, and is indicated for use under the trade name Precedex®. At lower doses, dexmedetomidine has now been observed to provide analgesic and anxiolytic benefits, without risk of respiratory depression.

The use of a transdermal dosage form with dexmedetomidine provides a non-invasive mechanism for extended drug uptake. This extended uptake facilitates a prolonged duration of action, with potential benefit to chronic pain patients. Dexmedetomidine has the potential to control pain without additional sedation or respiratory depression. Appropriate use should allow patients to limit discomfort, while maintaining their ability to take part in daily activities.

The first human use of the exemplary transdermal formulations described herein was designed to evaluate the safety, tolerability and pharmacokinetic properties of two transdermal formulations of dexmedetomidine identified herein as DEX-TD.02 and DEX-TD.03. Pharmacokinetic observations will be focused to evaluate the absorption of dexmedetomidine across human skin, and the impact of an escalated dose.

Study Objective.

The objective of the human study was to evaluate the safety, tolerability, and pharmacokinetics of single escalating doses of transdermal dexmedetomidine in healthy male and female subjects.

Investigational Plan.

The overall study design was a Phase 1, open-label, single-dose, dose escalation, crossover study in healthy subjects to investigate the safety, tolerability, and pharmacokinetics of two formulations of transdermal dexmedetomidine, namely DEX-TD.02 and DEX-TD.03. Healthy subjects between the ages of 18 and 50 years, inclusive, were screened for participation at one study site in Australia within 28 days before study drug administration. Medical history, physical examination, baseline laboratory testing, 12-lead electrocardiogram (ECG), pregnancy testing, vital sign measurements, and informed consent were completed during the screening visit. Prior to dosing in Period 1, study participants were randomly assigned to one of two study treatment sequences (see Table M). Each participant received a single dose of study medication in each study period according to assigned treatment sequence. Dosing periods were separated by a sufficient window to allow for pharmacokinetic analysis of plasma drug concentrations in the previous period (approximately 14 days).

TABLE M
Study Treatment Sequences

| | Study | | Study | | Study |
|---|---|---|---|---|---|
| Treatment | Period 1 | | Period 2 | | Period 3 |
| Sequence | (Left) | Washout | (Right) | Washout | (Left) |
| 1 (N = 3) | A | → | C | → | E |
| 2 (N = 3) | B | → | D | → | E |

During each period, subjects were to be confined from the evening before dosing (Day 1) to approximately 48 hours after dosing (Day 3). Subjects were required to shower in the morning before administration and were prohibited from showering or washing the area for 48 hours after administration. While confined, subjects received a standardized daily diet. Table N shows the medications, applied doses, and topical areas of administration in this study.

TABLE N
Study Treatments

| | Applied Amount | | |
|---|---|---|---|
| | (Dexmedetomidine | Administration | Route of |
| Treatment | dose) | Area | Administration |
| A | 0.25 g DEX-TD.02 | Abdomen | Transdermal |
| | (250 μg) | | |
| B | 0.25 g DEX-TD.03 | Abdomen | Transdermal |
| | (250 μg) | | |
| C | 0.5 g DEX-TD.02 | Abdomen—Not | Transdermal |
| | (500 μg) | Occluded | |
| D | 0.5 g DEX-TD.02 | Abdomen—Occluded | Transdermal |
| | (500 μg) | | |
| E | 1 g DEX-TD.02 | Abdomen—Not | Transdermal |
| | (1000 μg) | Occluded | |

For pharmacokinetic analyses, 16 blood samples were collected in each period from each subject following administration of a single dose. The collections were targeted for predose (Time 0) and at the following approximate times after dosing with study medication: 30, 60, and 90 minutes and 2, 3, 4, 6, 8, 10, 12, 18, 24, 30, 36, and 48 hours. The time after dosing began when the dose was applied to the subject. Safety assessments included monitoring of AEs, clinical laboratory tests, vital sign measurements, physical examinations, irritation assessments, and ECGs.

Rationale for Study Design and Control Groups.

This study evaluated the safety, tolerability, and pharmacokinetic properties of a known drug substance administered via an alternative route of delivery. Previous research by the inventors and applicant has demonstrated the safety of dexmedetomidine when administered by the intravenous and sublingual routes.

As previously described herein, preliminary studies by the inventors and applicant have been performed using DEX-TD formulations in animal models (canine and swine) to support this initial dosing in healthy human subjects. The dose escalation model used in this study was to identify and evaluate the absorption properties of DEX-TD.02 and DEX-TD.03 when applied to human skin, including how they may vary dependent upon the dose applied. The purpose of the crossover design was to compare the absorption of these two formulations in the same set of subjects. In one sub-group of subjects, the transdermal formulation was administered to an area of skin and promptly covered with a dressing (“occluded”). The dressing used was TEGADERM™ brand dressing (TEGADERM™ is a registered trademark of 3M Corporation). In the other sub-group of subjects, the transdermal formulation was administered to the skin and left uncovered (“non-occluded”). Based upon well-established experience with occluded administration, the inventors expected that the absorption would be higher in the subjects having a dressing applied over the transdermal formula skin application site (abdomen). However, surprisingly, the absorption of DEX-TD.02 was significantly higher for the sub-group where no dressing was applied over the skin application site. Further, the results indicate that no occlusion is necessary or desirable, making the composition of at least DEX-TD.02 a true “patchless” transdermal (i.e., no dressing, no need for a patch to contain the formulation). The formulation is stable when applied to the skin and is readily absorbed with no need for dressings or other coverings. Due to the pharmacokinetic focus of this study, no control population was necessary.

Following review of the pharmacokinetic results in Period 1, it was decided only to dose formulation DEX-TD.02 during clinical study Period 2. This was in part due to an apparent lower absorption from DEX-TD.03. Since the study sought high absorption in order to provide systemic absorption of dexmedetomidine, DEX-TD.02 was more appropriate for further clinical testing in this study. However, DEX-TD.03, given its lower absorption rate and very low systemic absorption, may be much more appropriate for other pain management uses such as treatment of localized skin and/or subdural neuropathies. Notably, no sedation was observed or reported for any of the subjects treated with either formulation in study Period 1.

### Clinical Study Period 2

Objective.

The objective of clinical study Period 2 was to calculate the steady-state plasma concentration-time profile resulting from once-daily and twice-daily topical application of a 0.5-gram dose of dexmedetomidine of formulation DEX-TD.02, using data generated from the applicant's single ascending-dose study (Period 1, (REC-10-006)).

Study Design.

Dexmedetomidine plasma concentration-time data from clinical study # REC-10-006 were used in predicting the steady-state concentration-time profiles resulting from once-daily (QD) and twice-daily (BID) topical application of a 0.25, 0.5, and 1-gram dose. The data from the following 3 subjects were used: 1001, 1004, and 1005. The three subjects participated in the following treatment arms: Treatment A (0.25-gram dose), Treatment C (0.5-gram dose), and Treatment E (1.0-gram dose). Clinical study # REC-10-006 was a single ascending-dose study with multiple treatment arms (Treatments A, B, C, D and E). Plasma samples for drug analysis were taken at pre-dose (0 hour) and at the following specified times after dosing: 0.5, 1, 1.5, 2, 3, 4, 6, 8, 10, 12, 18, 24, 30, 36, and 48 hours. The lower limit of quantification of the bioanalytical assay was 0.02 ng/mL. Notably, no sedation was observed or reported for any of the subjects treated with either formulation in study Period 2.

Methodology and Analysis.

All pharmacokinetic parameters were analyzed using compartmental analysis. Dexmedetomidine plasma concentration-time data from Period 1 (REC-10-006) were used in predicting the steady-state concentration-time profiles resulting from once-daily (QD) and twice-daily (BID) topical application of 0.25-, 0.5- and 1.0-gram doses. The data used were from three subjects (Identifiers 1001, 1004, and 1005) who participated in treatment arms A (0.25-gram dose), C (0.5-gram dose), and E (1.0-gram dose). Blood samples for drug analysis were taken at pre-dose and at the following specified times after dosing: 0.25, 0.5, 0.75, 1, 1.5, 2, 2.5, 3, 4, 5, 6, 7, 8, 12, 24, 48, and 72 hours. A major portion (67%) of the plasma concentrations was below the quantifiable level of 0.02 ng/mL; therefore, the PK parameters were calculated using the more sophisticated computational method of nonlinear-mixed-effect models (i.e., population pharmacokinetics [PopPK]). The nonlinear-mixed-effect model was carried out using the software Monolix, Version 3.1 (Institut National de Recherche en Informatique et en Automatique [INRIA]). Once the PK parameters were calculated and the single-dose concentrations were generated, the data were imported into WinNonlin™, Version 5.2 (WinNonlin™ Copyright ©2008, Pharsight Corporation), a nonparametric superposition was carried out to predicted the steady-state plasma concentration-time profiles resulting from QD and BID dosing.

Calculation of the Pharmacokinetic Parameters from Single-Dose Data.

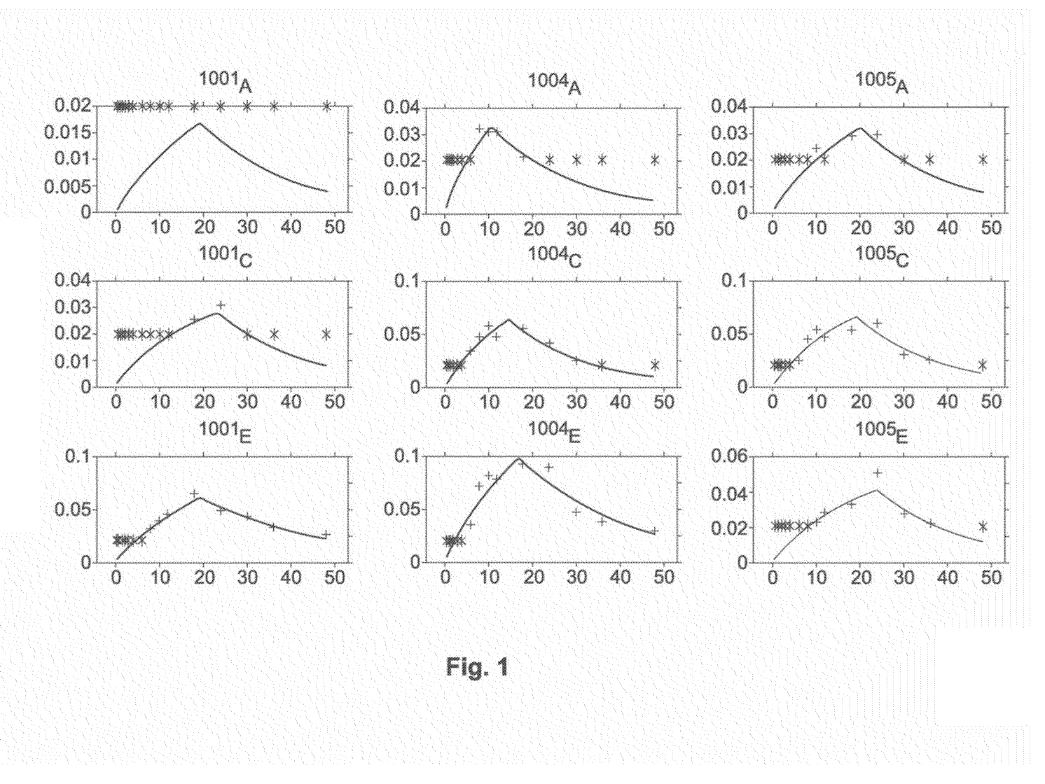

As stated previously, the PK parameters were calculated using nonlinear-mixed-effect model using the software MonoLix 3.1. Plasma concentrations of dexmedetomidine from the three subjects (1001, 1004 and 1005) who participated in treatment arms A (0.25-gram dose), C (0.5-gram dose) and E (1.0-gram dose) were fitted to a linear one-compartment model with zero-order absorption. The fitted and observed concentrations are shown in FIG. 1. In FIG. 1, note: number is the subject identifier (e.g. 1001, 1004 or 1005); subscript (e.g. A, C, E) is the treatment: A (0.25-gram dose), C (0.5-gram dose) or E (1.0-gram dose); plus symbols (+) are the measurable concentrations of dexmedetomidine above the limit of quantification; and Asterisk (*) symbols are concentrations of dexmedetomidine below limit of quantification (0.02 ng/mL); and solid line constitutes best fitted PK curve. Inspection of the fitted and observed concentration-time profiles (FIG. 1) indicated that the one-compartment with zero-order absorption (solid line) adequately describes both the quantifiable concentrations of dexmedetomidine (plus symbols) and is consistent with those samples that were deemed to be below the quantifiable limit (BQL) (asterisk symbols).

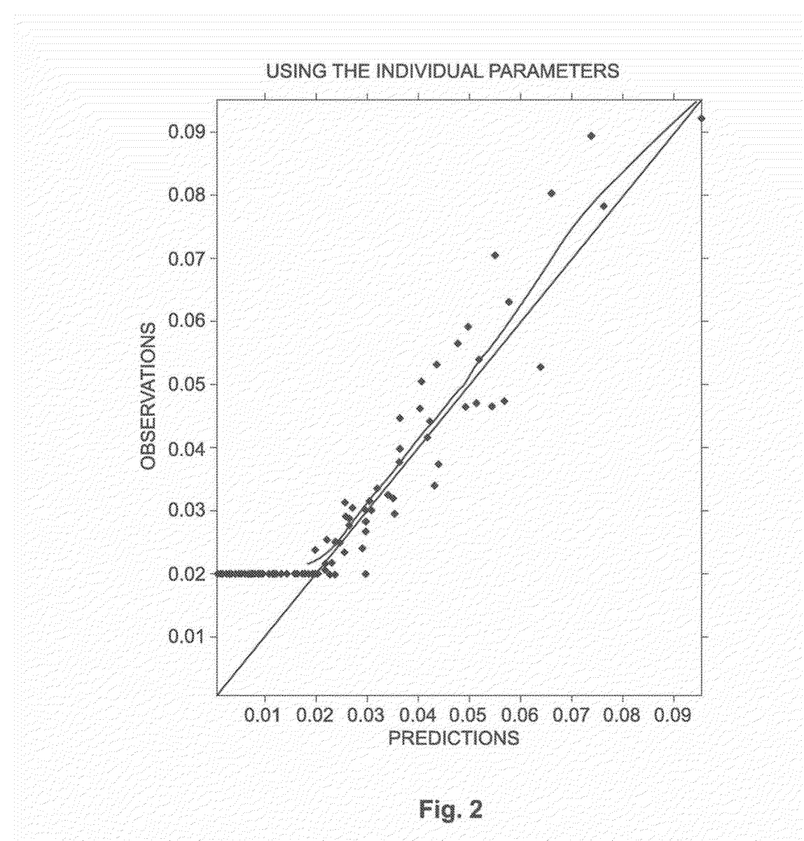

Overall, there was good agreement between the model and the observed values from subject dosing, as shown in FIG. 2. FIG. 2 shows that the predicted values (from the model) are in close agreement with the observed values as indicated by the scattering which is close to the theoretical line for observed values that are equal to predicted values and that most of the BQL values are constant with the values from the model. For FIG. 2, note: diamonds are the measurable concentrations above limit of quantification; asterisks are the measurable concentrations below limit of quantification (0.02 ng/mL); straight line is the line of unity where observed=predicted concentrations; and curved line is a trend line for the plot points.

The derived pharmacokinetic parameters from the fitting are presented in Table P1. Dermal application of the dexmedetomidine appeared to be absorbed at a constant rate (similar to constant infusion) over approximately a 20-hour period with a half-life of about 15 hours. An accumulation factor of about 2.2× and 1.5× was calculated for BID and QD dosing, respectively.

TABLE P1
Calculated Pharmacokinetic Parameters
from Clinical Study 1 (REC-10-06)

| | | Zero- | | | | Accumulation | |
|---|---|---|---|---|---|---|---|
| | | Order | V/F* | | half- | Factor | |
| Treat- | | Input | (10⁶ | kₑₗ^{‡} | life | Dosing | Dosing |
| ment | ID | Time (hr) | Liters) | (hr⁻¹) | (hr) | BID | QD |
| A | 1001 | 22 | 21 | 0.055 | 13 | 2.1 | 1.4 |
| (0.25 | 1004 | 11 | 6 | 0.057 | 12 | 2.0 | 1.3 |
| gram) | 1005 | 20 | 5 | 0.048 | 14 | 2.3 | 1.5 |
| | Mean | 18 | 11 | 0.05 | 13 | 2.1 | 1.4 |
| C | 1001 | 23 | 11 | 0.047 | 15 | 2.3 | 1.5 |
| (0.5 | 1004 | 15 | 5 | 0.060 | 12 | 2.0 | 1.3 |
| gram) | 1005 | 18 | 5 | 0.055 | 13 | 2.1 | 1.4 |
| | Mean | 19 | 7 | 0.05 | 13 | 2.1 | 1.4 |
| E | 1001 | 19 | 12 | 0.035 | 20 | 2.9 | 1.8 |
| (1.0 | 1004 | 17 | 7 | 0.043 | 16 | 2.5 | 1.5 |
| gram) | 1005 | 24 | 14 | 0.054 | 13 | 2.1 | 1.4 |
| | Mean | 20 | 11 | 0.04 | 16 | 2.5 | 1.6 |

*V/F: Volume of distribution divided by the fraction of drug absorbed
^{‡}kₑₗ: Apparent first-order elimination rate constant

Calculation of Steady-State Concentrations Resulting from Both BID and QD Dosing.

As stated previously, once the PK parameters were calculated and the single-dose concentrations were generated, the data were imported into WinNonlin™, Version 5.2 (WinNonlin™ Copyright ©2008, Pharsight Corporation). Nonparametric superposition was carried out to predict the steady-state plasma concentration-time profiles resulting from QD and BID dosing. Nonparametric superposition assumes that each dose of a drug acts independently of every other dose and that the rate and extent of absorption and average systemic clearance are the same for each dosing interval. Linear pharmacokinetics applies so that a change in dose during the multiple-dosing regimen can be accommodated.

In order to predict the drug concentrations resulting from multiple doses, a complete characterization of the concentration-time profile after a single dose administration is necessary. That is, it is necessary to know C(tᵢ) at sufficient time points tᵢ, (i=1, 2, . . . , n), to characterize the drug absorption and elimination process. Two assumptions about the data are required: independence of each dose effect, and linearity of the underlying pharmacokinetics. The former assumes that the effect of each dose can be separated from the effects of other doses. The latter, linear pharmacokinetics, assumes that changes in drug concentration will vary linearly with dose amount.

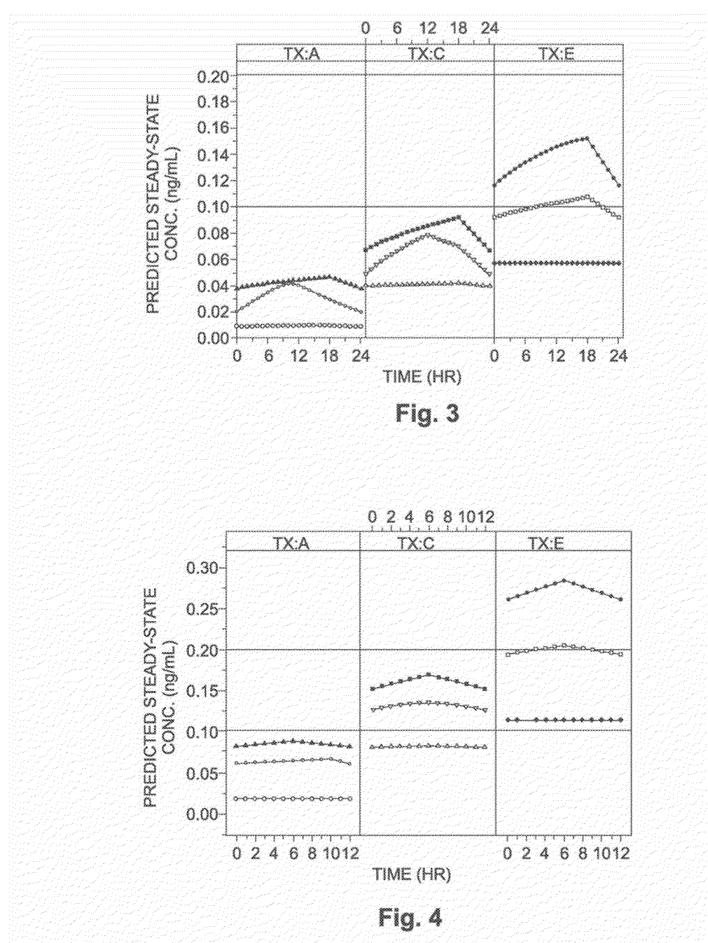

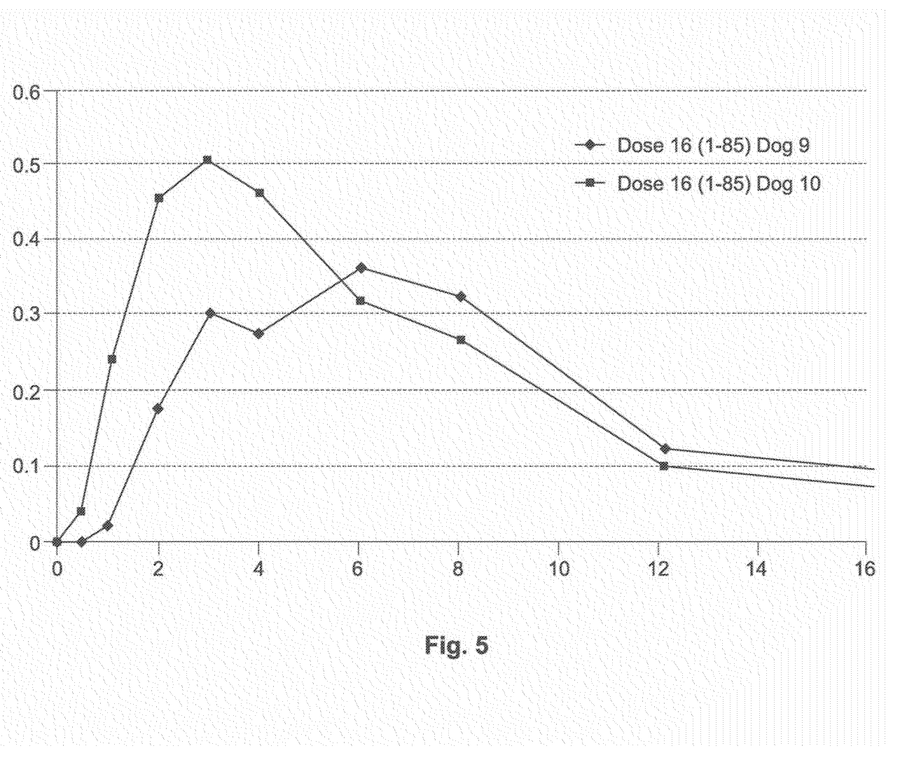

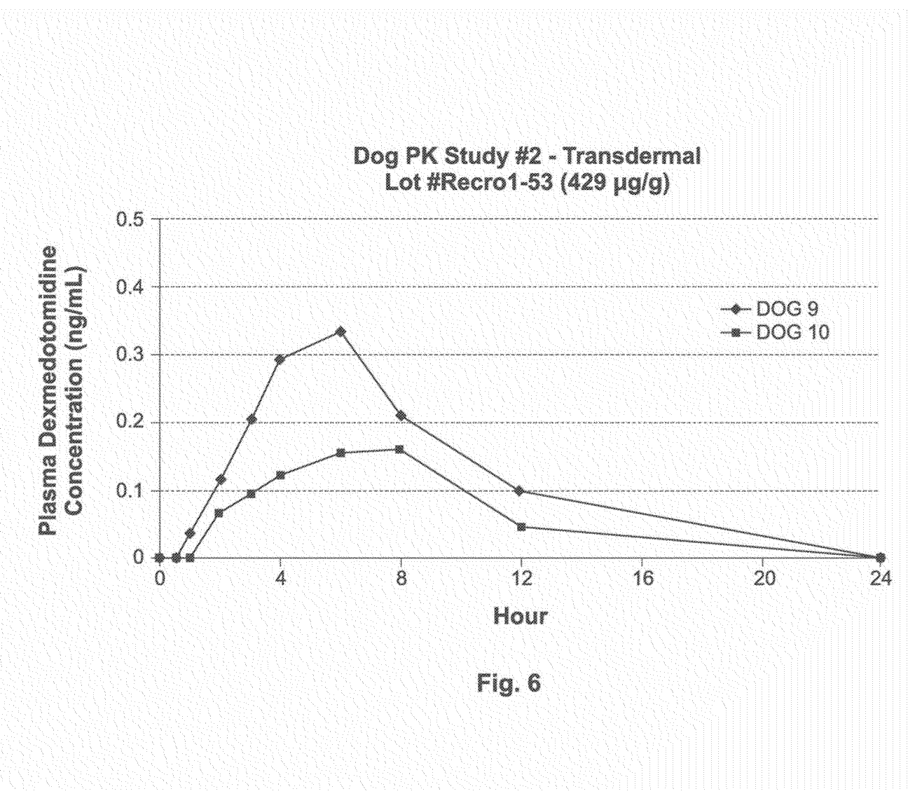

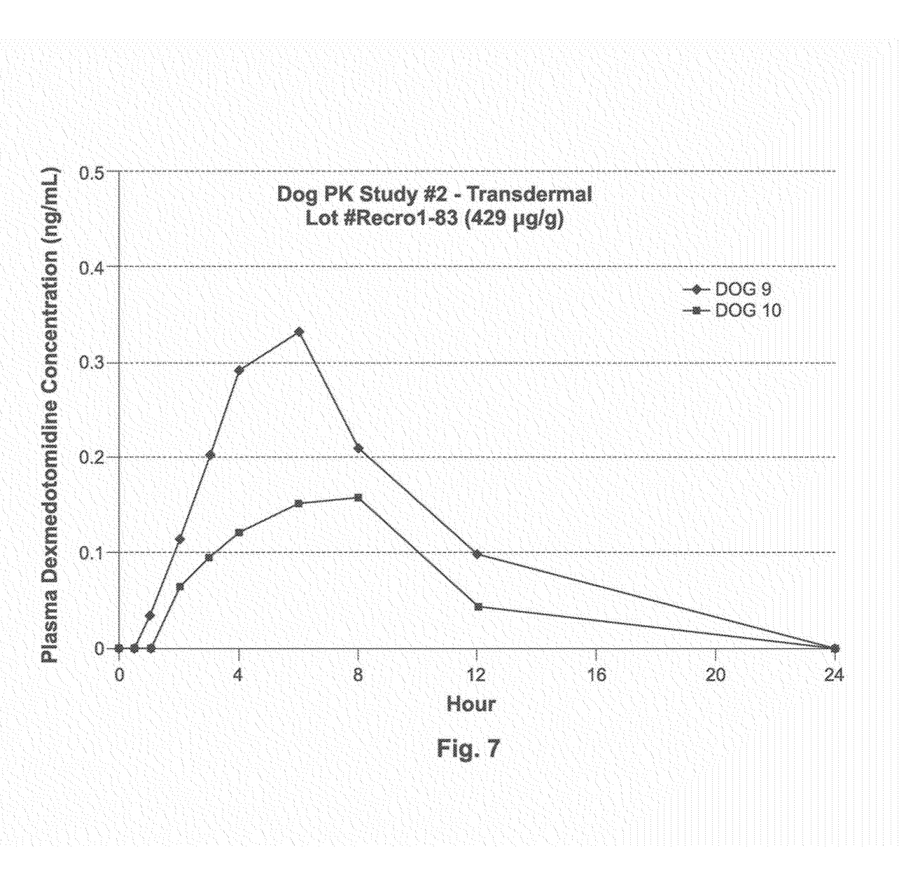

The predicted plasma concentration-time curves at steady-state after once daily doses of 0.25 (tx A), 0.5 (tx C) and 1.0 (tx E) grams are shown in FIG. 3. The predicted steady-state plasma concentration-time curves after twice-daily dosing are shown in FIG. 4. For both dosing regimens (QD and BID), the resulting plasma concentration-time curves at steady-state are fairly flat. This is due to the prolonged zero-order diffusion of drug from the dermis to the systemic circulation and long half-life. The predicted steady-state plasma concentrations for BID and QD dosing regimens are listed in Table Q2 and Table R3, respectively.

TABLE Q2
Steady-State Plasma Concentration (ng/mL) Resulting from Twice-Daily Dosing

| | | Time (hr) | | | | | | | | | | | | |
|---|---|---|---|---|---|---|---|---|---|---|---|---|---|---|
| Treatment | ID | 0 | 1 | 2 | 3 | 4 | 5 | 6 | 7 | 8 | 9 | 10 | 11 | 12 |
| A | 1001 | 0.019 | 0.019 | 0.019 | 0.020 | 0.020 | 0.020 | 0.020 | 0.020 | 0.020 | 0.020 | 0.019 | 0.019 | 0.019 |
| (0.25 gram) | 1004 | 0.062 | 0.063 | 0.063 | 0.064 | 0.064 | 0.065 | 0.065 | 0.065 | 0.066 | 0.066 | 0.066 | 0.064 | 0.062 |
| | 1005 | 0.082 | 0.083 | 0.085 | 0.086 | 0.087 | 0.087 | 0.088 | 0.087 | 0.087 | 0.086 | 0.085 | 0.083 | 0.082 |
| | Mean | 0.054 | 0.055 | 0.056 | 0.056 | 0.057 | 0.057 | 0.058 | 0.058 | 0.057 | 0.057 | 0.057 | 0.056 | 0.054 |
| C | 1001 | 0.081 | 0.082 | 0.082 | 0.082 | 0.082 | 0.083 | 0.083 | 0.083 | 0.082 | 0.082 | 0.082 | 0.082 | 0.081 |
| (0.5 gram) | 1004 | 0.127 | 0.129 | 0.131 | 0.132 | 0.134 | 0.135 | 0.136 | 0.135 | 0.134 | 0.132 | 0.130 | 0.129 | 0.127 |
| | 1005 | 0.152 | 0.156 | 0.159 | 0.162 | 0.165 | 0.167 | 0.170 | 0.167 | 0.165 | 0.162 | 0.159 | 0.155 | 0.152 |
| | Mean | 0.120 | 0.122 | 0.124 | 0.125 | 0.127 | 0.128 | 0.129 | 0.128 | 0.127 | 0.125 | 0.124 | 0.122 | 0.120 |
| E | 1001 | 0.195 | 0.197 | 0.198 | 0.200 | 0.202 | 0.204 | 0.205 | 0.204 | 0.202 | 0.200 | 0.199 | 0.197 | 0.195 |
| (1.0 gram) | 1004 | 0.262 | 0.266 | 0.270 | 0.274 | 0.278 | 0.281 | 0.284 | 0.281 | 0.277 | 0.274 | 0.270 | 0.266 | 0.262 |
| | 1005 | 0.114 | 0.114 | 0.115 | 0.115 | 0.115 | 0.115 | 0.115 | 0.115 | 0.115 | 0.115 | 0.115 | 0.115 | 0.114 |
| | Mean | 0.190 | 0.192 | 0.194 | 0.196 | 0.198 | 0.200 | 0.202 | 0.200 | 0.198 | 0.196 | 0.194 | 0.192 | 0.190 |

TABLE R3
Steady-State Plasma Concentration (ng/mL) Resulting from Once-Daily Dosing

| | | Time (hr) | | | | | | | | | | | | |
|---|---|---|---|---|---|---|---|---|---|---|---|---|---|---|
| Treatment | ID | 0 | 2 | 4 | 6 | 8 | 10 | 12 | 14 | 16 | 18 | 20 | 22 | 24 |
| A | 1001 | 0.009 | 0.009 | 0.009 | 0.010 | 0.010 | 0.010 | 0.010 | 0.010 | 0.010 | 0.010 | 0.010 | 0.010 | 0.009 |
| (0.25 gram) | 1004 | 0.021 | 0.026 | 0.031 | 0.035 | 0.039 | 0.043 | 0.041 | 0.037 | 0.033 | 0.029 | 0.027 | 0.024 | 0.021 |
| | 1005 | 0.038 | 0.040 | 0.041 | 0.042 | 0.043 | 0.044 | 0.044 | 0.045 | 0.046 | 0.047 | 0.044 | 0.041 | 0.038 |
| | Mean | 0.023 | 0.025 | 0.027 | 0.029 | 0.031 | 0.032 | 0.032 | 0.031 | 0.030 | 0.029 | 0.027 | 0.025 | 0.023 |
| C | 1001 | 0.040 | 0.040 | 0.041 | 0.041 | 0.041 | 0.041 | 0.041 | 0.042 | 0.042 | 0.042 | 0.041 | 0.041 | 0.040 |
| (0.5 gram) | 1004 | 0.048 | 0.055 | 0.061 | 0.066 | 0.071 | 0.075 | 0.078 | 0.075 | 0.073 | 0.070 | 0.063 | 0.056 | 0.049 |
| | 1005 | 0.067 | 0.071 | 0.075 | 0.078 | 0.081 | 0.083 | 0.086 | 0.088 | 0.090 | 0.092 | 0.084 | 0.075 | 0.067 |
| | Mean | 0.052 | 0.056 | 0.059 | 0.061 | 0.064 | 0.067 | 0.068 | 0.068 | 0.068 | 0.068 | 0.063 | 0.057 | 0.052 |
| E | 1001 | 0.092 | 0.094 | 0.096 | 0.098 | 0.100 | 0.102 | 0.103 | 0.104 | 0.106 | 0.108 | 0.102 | 0.097 | 0.092 |
| (1.0 gram) | 1004 | 0.116 | 0.123 | 0.128 | 0.133 | 0.138 | 0.142 | 0.146 | 0.147 | 0.149 | 0.152 | 0.140 | 0.128 | 0.116 |
| | 1005 | 0.057 | 0.057 | 0.058 | 0.057 | 0.058 | 0.058 | 0.057 | 0.057 | 0.058 | 0.058 | 0.058 | 0.057 | 0.057 |
| | Mean | 0.088 | 0.091 | 0.094 | 0.096 | 0.098 | 0.100 | 0.102 | 0.103 | 0.104 | 0.106 | 0.100 | 0.094 | 0.088 |

Period 2 Results and Conclusions.

FIG. 3 and FIG. 4 show the predicted steady state plasma concentrations over time in adult humans based upon the clinical study using DEX-TD.02, in once-daily and twice-daily dose regimens. Recall that the target steady-state concentrations of dexmedetomidine for use as an analgesic without sedation in this study are in the range of 0.1 to 0.2 ng/mL. Therefore, as FIGS. 3 and 4 clearly illustrate, this can be achieved with BID dosing of either a 0.5-gram dose (which will achieve the lower part of the range) or 1.0-gram dose (which will result in concentration in the upper range). In this example, the desired steady-state concentrations will be predicted to occur on or after the 4^{th} day of multiple dosing. Based on this model, once-daily dosing remains a challenge, since once daily dosing using DEX-TD.02 and DEX-TD.03 did not quickly yield steady-state plasma concentrations reaching the target range (0.1 to 0.2 ng/mL) with either the 0.5-gram dose or 1.0-gram dose. Again, using DEX-TD.02 or a similar formulation, steady-state concentrations will be reached by at least the 4^{th} day of multiple dosing. However, with knowledge and information herein and contemplated by the inventors, these and other topical formulations may be shown to transdermally absorb and accomplish the desired target steady state plasma concentrations of dexmedetomidine in mammals, whether in a shorter period of time or through alternate dosing regimens. For example, dosing to a desired steady state can be accomplished using a combination of a rapidly absorbing sublingual, transmucosal, and/or rapid absorbing transdermal composition (e.g. including a fast absorbing solvent carrier such as DMSO, alcohols, and other pharmaceutically compatible carriers and formulations), in combination with the sustained, controlled, prolonged release and absorption dexmedetomidine transdermal formulations described herein.

While this description is made with reference to exemplary embodiments, it will be understood by those skilled in the art that various changes may be made and equivalents may be substituted for elements thereof without departing from the scope. In addition, many modifications may be made to adapt a particular situation or material to the teachings hereof without departing from the essential scope. Also, in the description there have been disclosed exemplary embodiments and, although specific terms may have been employed, they are unless otherwise stated used in a generic and descriptive sense only and not for purposes of limitation, the scope of the claims therefore not being so limited. Moreover, one skilled in the art will appreciate that certain steps of the methods discussed herein may be sequenced in alternative order or steps may be combined. Therefore, it is intended that the appended claims not be limited to the particular embodiment disclosed herein.

1. A method of treating or preventing pain comprising
applying to the skin of the mammal a composition comprising a dosage of dexmedetomidine or a pharmaceutically acceptable salt or pro-drug thereof in a pharmaceutically acceptable vehicle,
wherein the dexmedetomidine or pharmaceutically acceptable salt or pro-drug thereof is absorbed through the skin and produces analgesia without sedation.

2. The method according to claim 1, wherein said dosage of dexmedetomidine or pharmaceutically acceptable salt thereof is between about 0.05 μg/kg and about 15 mcg/kg, and wherein said mammal is a human.

3. The method according to claim 2, wherein said mammal is a human, and said transdermal dosage of dexmedetomidine or pharmaceutically acceptable salt thereof is between about 100 μg and about 1500 μg.

4. The method according to claim 3, wherein the plasma cₘₐₓ of dexmedetomidine upon absorption from transdermal application and into the systemic circulatory system of said human is less than about 0.30 ng/mL.

5. The method according to claim 2, wherein said applying step comprises topically applying said pharmaceutical composition to the skin of at least one of the legs, arms, abdomen, chest, groin, neck, back, and shoulders.

6. The method according to claim 5, wherein said applying step comprises applying a cream, lotion, or gel of the pharmaceutical composition to said skin of said mammal.

7. The method according to claim 6, wherein said applying step does not require covering of the skin with a dressing after administration of said pharmaceutical composition onto the skin of said mammal.

8. The method according to claim 2, wherein, during at least the 6 hours immediately after applying said pharmaceutical composition, the resting mean arterial blood pressure of the human varies by no more than about 20 mmHg.

9. The method according to claim 2, wherein, during the at least the 6 hours immediately after applying said pharmaceutical composition, said human is able to remain awake and to respond to commands, and said human is alert and oriented.

10. The method according to claim 1, wherein said dexmedetomidine or pharmaceutically acceptable salt thereof is co-administered with one or more other analgesics.

11. The method according to claim 10, wherein said pharmaceutical composition is administered intermittently to treat breakthrough pain that is inadequately controlled by said one or more other analgesics.

12. The method according to claim 2, wherein said pain is idiopathic pain.

13. The method according to claim 12, wherein said idiopathic pain is selected from the group consisting of neuralgia, myalgia, hyperalgia, hyperpathia, neuritis, and neuropathy.

14. The method according to claim 2, wherein said pain is associated with or caused by cancer, viral infection, physical trauma, arthritis, headache, or lower back pain.

15. The method according to claim 14, wherein said physical trauma is associated with or caused by surgery, a burn, or blunt force trauma.

16. A method of treating or preventing pain comprising
applying to a skin membrane of a mammal a pharmaceutical composition comprising dexmedetomidine, or a pharmaceutically acceptable salt or pro-drug thereof, in a pharmaceutically acceptable vehicle,
wherein said dexmedetomidine, or said pharmaceutically acceptable salt or pro-drug thereof, is absorbed through said skin and produces analgesia without sedation.

17. A method of treating or preventing pain comprising
administering to the skin of a mammal a systemically absorbed pharmaceutical composition comprising dexmedetomidine, or a pharmaceutically acceptable salt or pro-drug thereof, in an amount effective to treat or to prevent pain in said mammal upon administration,
wherein said pharmaceutical composition provides a physiologically active amount of dexmedetomidine into the systemic circulatory system of said mammal at a rate that produces an analgesic effect without sedation within at least 6 hours of administration.

18. An analgesic pharmaceutical composition comprising dexmedetomidine, or a pharmaceutically acceptable salt or pro-drug thereof, in a pharmaceutically acceptable vehicle, said pharmaceutical composition being configured and adapted for topical administration to a mammal by applying said analgesic pharmaceutical composition to the skin of said mammal.

19. The analgesic pharmaceutical composition according to claim 18, wherein said analgesic pharmaceutical composition is configured and adapted for topical administration by applying said composition to the skin more that once daily.

20. The analgesic pharmaceutical composition according to claim 18, wherein said analgesic pharmaceutical composition is configured and adapted for topical administration by applying said composition to the skin of at least one of the legs, arms, abdomen, chest, groin, neck, back, and shoulders of said mammal.

21. The analgesic pharmaceutical composition according to claim 18, wherein said pharmaceutically acceptable salt of dexmedetomidine is dexmedetomidine hydrochloride, and wherein said vehicle is selected from at least one of creams, gels, and suspensions.

22. The analgesic pharmaceutical composition according to claim 21, wherein the composition does not require an occlusion or covering after administration onto the skin of a mammal.

23. The analgesic pharmaceutical composition according to claim 18, wherein said analgesic pharmaceutical composition is packaged in a dispensing device.

24. An apparatus for treating or preventing pain, said apparatus comprising
an analgesic pharmaceutical composition comprising dexmedetomidine, or a pharmaceutically acceptable salt or pro-drug thereof, in a pharmaceutically acceptable vehicle, and
a dispensing device that contains and dispenses said analgesic pharmaceutical composition.

25. The apparatus of claim 24, wherein the apparatus includes an applicator configured and arranged for conveying the composition from the apparatus to the skin of a mammal.
